(19) United States
(12) Patent Application Publication
(10) Pub. No.: US 2023/0372096 A1
(43) Pub. Date: Nov. 23, 2023

(54) Paravalvular Leak Protection for Balloon Expandable Valves

(71) Applicant: St. Jude Medical, Cardiology Division, Inc., St. Paul, MN (US)

(72) Inventors: Tracee Eidenschink, Wayzata, MN (US); Peter J. Ness, Minneapolis, MN (US); Daniel J. Klima, Andover, MN (US)

(73) Assignee: St. Jude Medical, Cardiology Division, Inc., St. Paul, MN (US)

(21) Appl. No.: 18/309,077

(22) Filed: Apr. 28, 2023

Related U.S. Application Data
(60) Provisional application No. 63/343,492, filed on May 18, 2022.

(51) Int. Cl. A61F 2/24

(52) U.S. Cl. CPC A61F 2/2433; A61F 2/2418; A61F 2250/0039

(57) ABSTRACT

A prosthetic heart valve system includes a balloon expandable prosthetic heart valve and a delivery catheter. The prosthetic heart valve has an inflow end portion, an outflow end portion, and a center portion. The delivery catheter has a balloon assembly on a distal end portion of the delivery catheter having a proximal portion, a distal portion, and a center portion. In a delivery condition of the system, the prosthetic heart valve is crimped over the balloon while the balloon assembly is deflated. In a deployment condition of the system, the balloon assembly is inflated so that the distal portion of the balloon assembly has a diameter that is larger than a diameter of the center portion of the balloon assembly, and so that the outflow end portion of the prosthetic heart valve flares radially outwardly relative to the center portion of the prosthetic heart valve

### CROSS-REFERENCE TO RELATED APPLICATIONS

This application claims priority to the filing date of U.S. Provisional Patent Application No. 63/343,492, filed May 18, 2022, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets, and may also include a cuff or skirt. The cuff may be disposed on the stent' s interior or luminal surface, its exterior or abluminal surface, and/or on both surfaces. A cuff helps to ensure that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When a prosthetic heart valve expands into the native annulus of the valve being replaced, it may be difficult to obtain a perfect seal between the outside of the prosthetic heart valve and the inner perimeter of the native valve annulus. As noted above, if a perfect seal does not occur, blood may flow through any space or gaps between the outside of the prosthetic heart valve and the inside of the native valve annulus. Thus, even if the valve assembly of the prosthetic heart valve works perfectly and no blood regurgitates through the valve assembly, blood regurgitation may occur via PV leak, reducing the effectiveness of the prosthetic heart valve replacement procedure. If a patient is a candidate for a prosthetic heart valve replacement procedure, that patient may be more likely than average to have a highly calcified native valve annulus. Such calcification may make it difficult to achieve a perfect seal with a prosthetic heart valve deployed therein. Some prosthetic heart valve features, such as outer cuffs, may help to reduce the likelihood and/or severity if PV leak occurring. For balloon expandable prosthetic heart valves, certain modifications may be made, either to the balloon (including how the prosthetic heart valve crimps onto the balloon, or how the balloon expands the prosthetic heart valve) or to the prosthetic heart valve itself, to further reduce the likelihood and/or severity of PV leak occurring.

### BRIEF SUMMARY OF THE DISCLOSURE

According to one aspect of the disclosure, a prosthetic heart valve system includes a balloon expandable prosthetic heart valve and a delivery catheter. The prosthetic heart valve has an inflow end portion, an outflow end portion, and a center portion between the inflow end portion and the outflow end portion. The delivery catheter has a balloon assembly on a distal end portion of the delivery catheter, the balloon assembly having a proximal portion, a distal portion, and a center portion between the proximal portion and the distal portion. In a delivery condition of the system, the prosthetic heart valve is crimped over the balloon while the balloon assembly is deflated. In a deployment condition of the system, the balloon assembly is inflated so that the distal portion of the balloon assembly has a diameter that is larger than a diameter of the center portion of the balloon assembly, and so that the outflow end portion of the prosthetic heart valve flares radially outwardly relative to the center portion of the prosthetic heart valve.

According to another aspect of the disclosure, a method of implanting a prosthetic heart valve includes delivering a prosthetic heart valve to a native valve annulus while the prosthetic heart valve is crimped on a balloon assembly of a balloon catheter and the balloon assembly is deflated. The method also includes inflating the balloon assembly to expand the prosthetic heart valve so that a center portion of the prosthetic heart valve contacts the native valve annulus and so that an inflow end portion of the prosthetic heart valve flares radially outwardly relative to the center portion of the prosthetic heart valve.

According to still another aspect of the disclosure, a prosthetic heart valve system includes a prosthetic heart valve, a delivery catheter, and a first outer cuff. The prosthetic heart valve includes a balloon expandable stent and a prosthetic valve assembly mounted within the stent. The delivery catheter has a balloon on a distal end portion of the delivery catheter, the system having (i) a delivery condition in which the prosthetic heart valve is crimped over the balloon while the balloon is deflated, (ii) a partially deployed condition in which the prosthetic heart valve is partially expanded and the balloon is partially inflated, and (iii) a fully deployed condition in which the prosthetic heart valve is fully expanded and the balloon is fully inflated. The first outer cuff is formed by a thread having a first end coupled to the balloon and a second end coupled to the stent, wherein in the delivery condition, the first outer cuff has a middle portion that wraps around the stent and wraps around a portion of the balloon positioned beyond a first end of the stent.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIG. 2A is a side view of a prosthetic heart valve crimped over a deflated balloon of a prosthetic heart valve delivery device.

FIG. 2B is an illustration of the balloon of FIG. 2A in an inflated condition.

FIG. 3A is a highly schematic illustration of a balloon expanding a prosthetic heart valve into a native aortic valve annulus.

FIGS. 3B-C illustrate a double balloon configuration to achieve the balloon shape of FIG. 3A.

FIG. 3D illustrates a single compliance, single balloon configuration to achieve the balloon shape of FIG. 3A.

FIGS. 3E-F illustrate a double compliance, single balloon configuration to achieve the balloon shape of FIG. 3A.

FIGS. 4A-D are schematic illustrations of different stages of a prosthetic heart valve being deployed, via balloon expansion, into a native valve annulus.

FIGS. 5A-C are schematic illustrations of different stages of a prosthetic heart valve being deployed via balloon expansion, according to another aspect of the disclosure.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation, but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

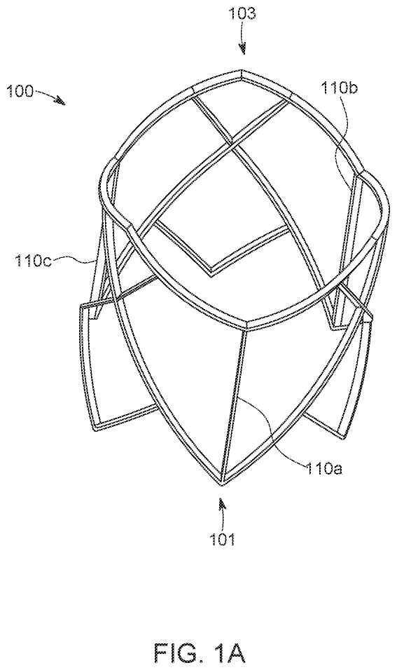

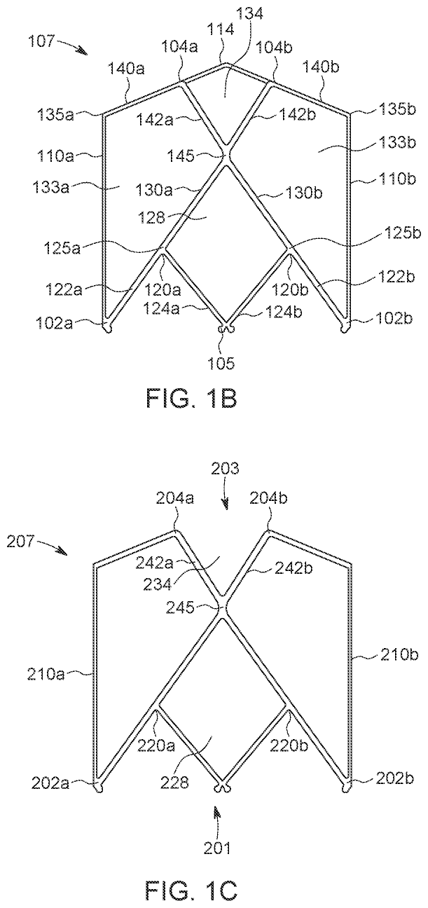

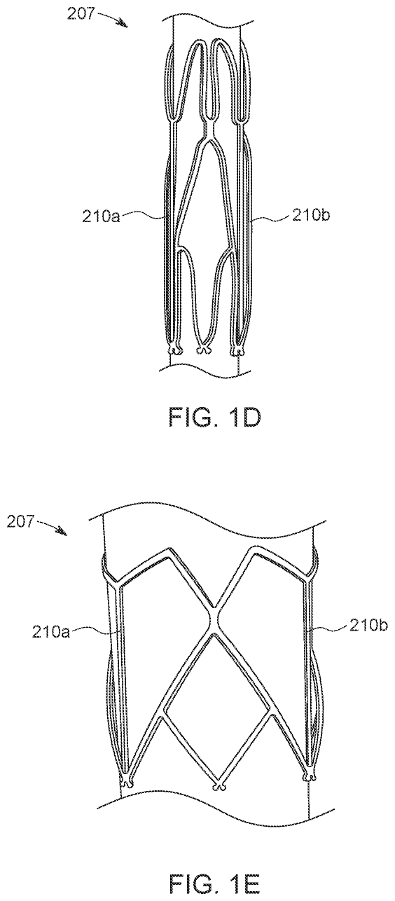

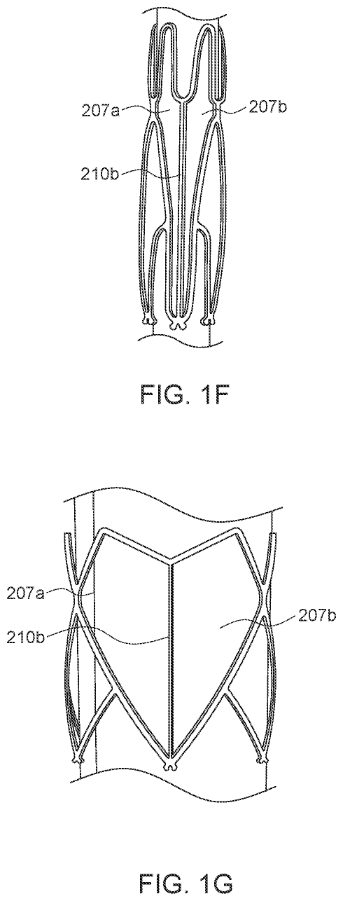

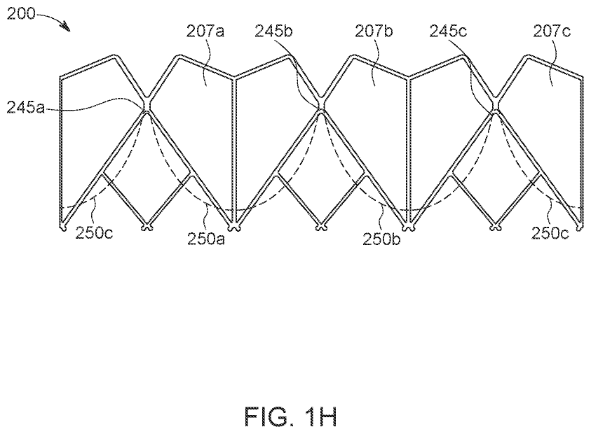

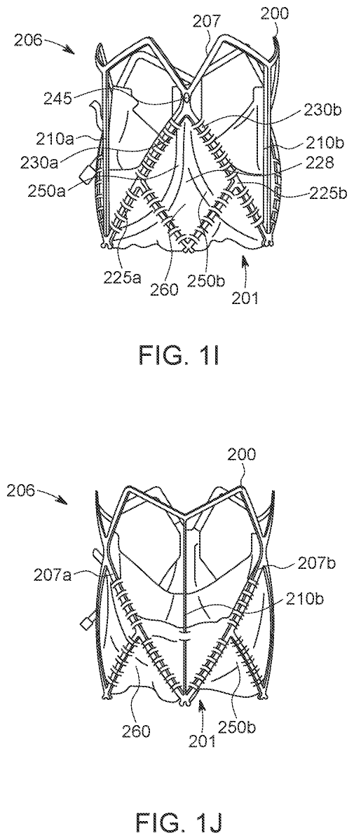

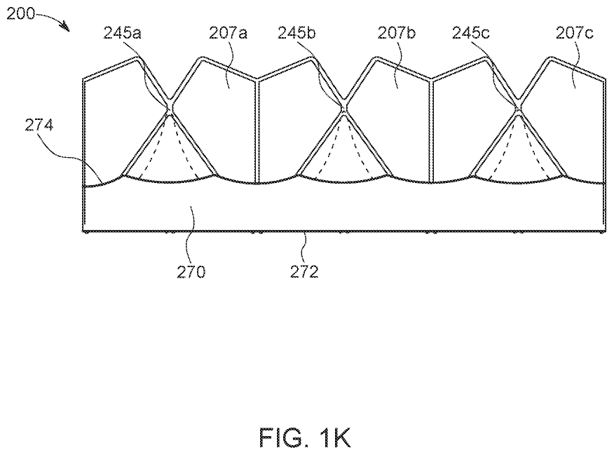

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100, and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 11 shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g. wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

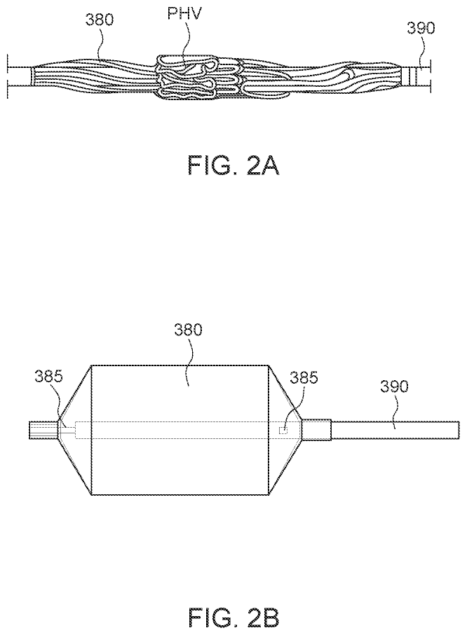

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

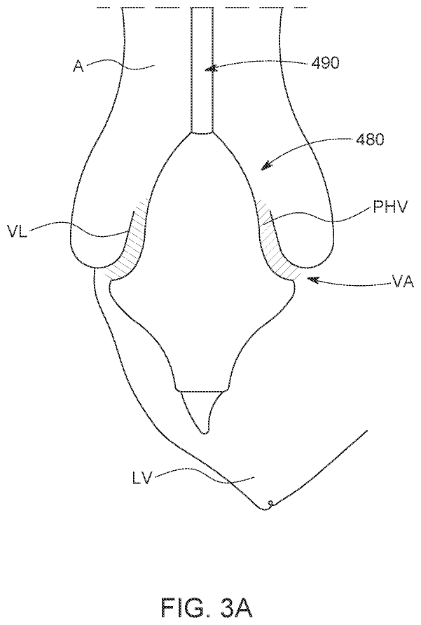

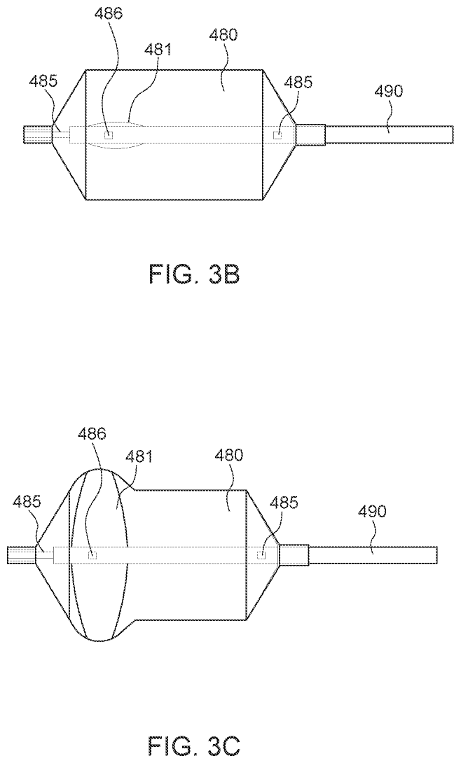

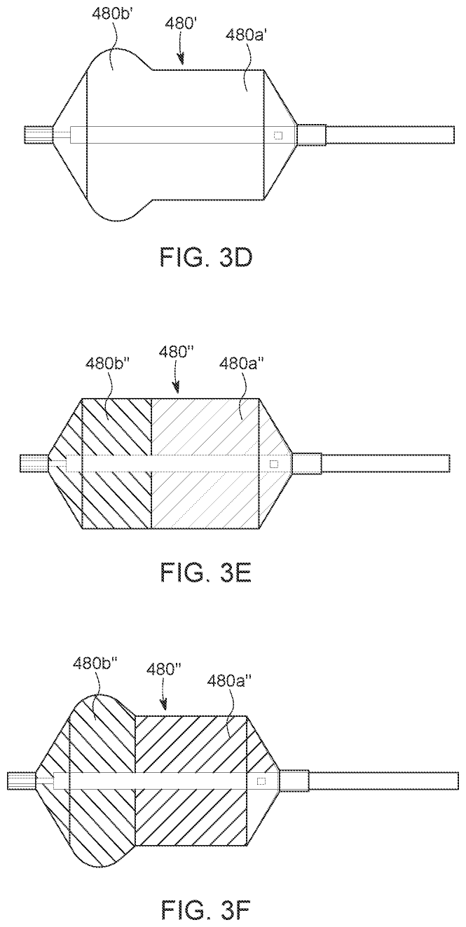

FIG. 3A is a highly schematic illustration of a prosthetic heart valve PHV being expanded, via a balloon 480, into the native annulus VA of an aortic valve of a patient. In this particular embodiment, a balloon catheter 490 has been advanced through the patient's femoral artery, around the aortic arch, through the aorta A, with a distal tip of the delivery device residing in the left ventricle LV and the prosthetic heart valve PHV being expanded via the balloon 480 into the native aortic valve annulus VA, with the prosthetic heart valve PHV pressing the native valve leaflets VL aside. The prosthetic heart valve PHV may be similar to that described in connection with FIGS. 1A-K, and may include an outer cuff similar to outer cuff 270, or may omit such an outer cuff. It should be understood that the concepts described in connection with this aspect of the disclosure may be used with other balloon expandable prosthetic heart valves taking different forms than those specifically described herein.

Still referring to FIG. 3A, as the balloon 480 expands and forces the prosthetic heart valve PHV to expand into the native aortic valve annulus VA, the inflow end of the prosthetic heart valve PHV is forced to flare radially outwardly relative to the outflow end of the prosthetic heart valve PHV. After deflating the balloon 480, the prosthetic heart valve PHV maintains the outward flare at its inflow end, which may create a better seal against the native aortic valve annulus VA and thus reduce the likelihood or severity of PV leak. Various options may be implemented to allow the balloon 480 to achieve the desired shape upon inflation that forces the prosthetic heart valve PHV to flare outwardly at its inflow end.

A first option to achieve the desired balloon shape shown in FIG. 3A is illustrated in FIGS. 3B-C. FIG. 3B is a side view of a distal end of a balloon catheter 490 that includes a balloon 480 inflatable via two ports 485 in the same manner as the balloon catheter of FIG. 2B. However, balloon catheter includes a secondary interior balloon 481, shown in a deflated condition in FIG. 3B and an inflated condition in FIG. 3C. The interior balloon 481 may be positioned over a separate inflation port 486 which may be isolated from the lumen(s) leading to inflation ports 485. The ends of the interior balloon 481 may be coupled to the shaft of balloon catheter 490 so that, as fluid flows into balloon 480 via ports 485, the interior balloon 481 does not inflate. The interior balloon 481 may be positioned within a distal end portion (e.g. a distal half, a distal third, or a distal quarter) along the length of the balloon 480. In use, when the prosthetic heart valve PHV is crimped over the deflated balloons 480, 481 of balloon catheter 490, the inflow end of the prosthetic heart valve PHV may be positioned over the interior balloon 481. During delivery, when the prosthetic heart valve PHV is positioned within the native valve annulus, the balloon 480 may be inflated first (as shown in FIG. 3B) by passing fluid through the balloon catheter 490 and through ports 485. Then, after balloon 480 is inflated, fluid may be passed through balloon catheter 490 (e.g. via a different lumen that does not lead to ports 485) and out of port 486 to inflate the interior balloon 481 to a larger diameter than the proximal side of the balloon 480. As the interior balloon 481 expands, it forces the distal end of the exterior balloon 480 to increase in diameter as well, forcing the inflow end of the prosthetic heart valve PHV to flare radially outwardly as shown in FIG. 3A. Although the second balloon 481 is shown and described as a balloon that is interior to balloon 480, other configurations may allow for similar results to be achieved. For example, the second balloon 481 could be positioned around an exterior of the first balloon 480, in generally the same axial location as shown in FIGS. 3B-C, with the exception that a portion of that exterior second balloon would be coupled to the shaft of the balloon catheter 490 in a location with a dedicated port so that the second balloon could be inflated separately after the first balloon is inflated. In that embodiment, the second balloon would not push the first balloon outwardly, but rather the first balloon would push the second balloon outwardly during inflation of the first balloon, and then the second balloon would be inflated in a separate second step to force the inflow end of the prosthetic heart valve PHV to flare outwardly as shown in FIG. 3A. Rather than positioning the first balloon 480 and second balloon 481 so that one is interior to the other, the second balloon 481 may be provided as an axially separate (e.g. sequential) balloon distal to the first balloon 480, so that there is little or no radial overlap between the two. In that embodiment, the second balloon 481 may be configured to expand to a greater diameter than the first balloon 480, and the balloons 480, 481 may be inflated at the same time, or sequentially with the first (in this case proximal) balloon 480 inflated first, and the second (in this case distal) balloon 481 being inflated second. Whether inflated sequentially or simultaneously, as the second balloon 481 inflates to a larger diameter than the first balloon 480, the inflow end of the prosthetic heart valve PHV will flare radially outwardly relative to the remainder of the prosthetic heart valve PHV, as shown in FIG. 3A.

Rather than use two balloon 480, 481 to achieve the flaring of the inflow end of the prosthetic heart valve PHV shown in FIG. 3A, a single balloon 480′ may instead be used to achieve the desired effect. For example, as shown in FIG. 3D the balloon 480′ may be formed from uniform material along the axial length of the balloon 480′, but the distal end 480b′ of the balloon 480′ may be shaped to have a slightly bulbous shape compared to the generally cylindrical shape of the proximal portion 480a′ (e.g. proximal three-quarters, proximal two-thirds, or proximal half) of the balloon 480′, with the distal bulbous shape 480b′ having a larger diameter when inflated than the proximal cylindrical portion 480a′.

Another option, as shown in FIGS. 3E-F, is to form the balloon 480″ to have a proximal portion 480a″ formed of a semi-compliant material, and a distal portion 480b″ formed of a less complaint material. With this configuration, as fluid is pushed into the balloon 480″, the two portions 480a″, 480b″ inflate until the balloon 480″ has a diameter that forces the prosthetic heart valve PHV to expand into the native valve annulus VA, as shown in FIG. 3E. However, as additional fluid is pushed into balloon 480″, the less compliant distal section 480b″ will continue to expand, while the more compliant proximal section 480a″ will tend not to significantly expand further. Thus, the inflow end of the prosthetic heart valve PHV will be forced to flare radially outwardly relative to other portions of the prosthetic heart valve PHV, as shown in FIG. 3A.

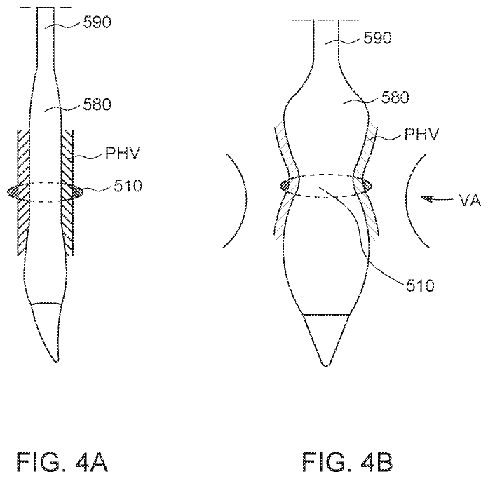

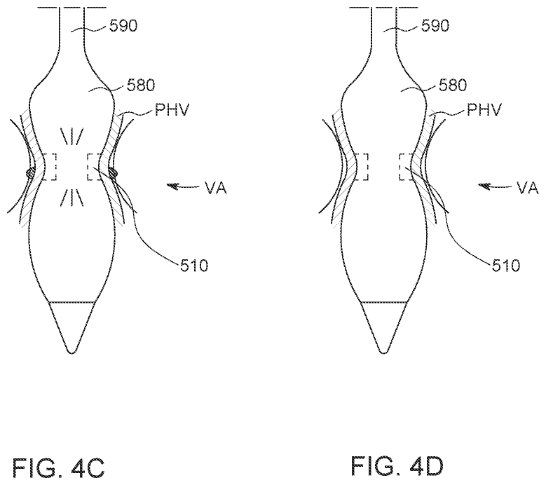

FIGS. 4A-D illustrate different stages of deployment of a prosthetic heart valve PHV using a balloon catheter 590 according to another embodiment of the disclosure. FIG. 4A illustrates the balloon catheter 590 in a position within or adjacent a native valve annulus VA (valve annulus shown in FIGS. 4B-D, but omitted in FIG. 4A), with a prosthetic heart valve PHV (which may be any balloon expandable prosthetic heart valve, including those described herein) crimped over the balloon 580 of the balloon catheter 590, the balloon 580 being in a deflated condition. As shown in FIG. 4A, a band 510 encircles the prosthetic heart valve PHV while it is over the balloon 580 in the illustrated condition. In the illustrated example, the band 510 circumscribes the prosthetic heart valve PHV and the balloon 580 at a position at or near the axial midpoint of each component. In other words, as illustrated, the prosthetic heart valve PHV is axially centered along the length of the balloon 580, and the band 510 is axially centered along the length of the balloon 580 and the prosthetic heart valve PHV. It should be understood that, in some embodiments, other positioning may be acceptable. In other words, the band 510 may be positioned offset to the axial center of the balloon 580 and/or the prosthetic heart valve PHV, although it may not be desirable for the band 510 to deviate significantly from the axial center of one or both of these components.

FIG. 4B illustrates the balloon 580 as it begins to expand. In the illustrated embodiment, balloon 580 may be formed of substantially uniform material and/or construction so that it is configured to expand into a generally cylindrical shape in the absence of constricting forces. The balloon 580 may begin expanding via fluid, such as a liquid (e.g. saline) being pushed through a lumen of the balloon catheter 590 that is in fluid communication with the interior volume of the balloon 580. It should be understood that, excluding the band 510, the balloon catheter 590 and balloon 580 may have any of the configurations described above, or any generic balloon catheter configuration. As the balloon 580 begins to expand, the band 510 limits both the balloon 580 and the prosthetic heart valve PHV from expanding at this axial position compared to other axial positions. This results in both the balloon 580 and prosthetic heart valve PHV beginning to have a general dog-bone shape, for example with the proximal and distal ends of the balloon 580 expanding to a larger diameter than the axial center of the balloon 580. Similarly, this results in the inflow and outflow ends of the prosthetic heart valve PHV initially expanding to a larger diameter than an axial center of the prosthetic heart valve PHV (or wherever the band 510 is positioned relative to the prosthetic heart valve PHV).

FIG. 4C illustrates the balloon 580 after the initial expansion begins, with the prosthetic heart valve PHV having been expanded with a general dog-bone shape so that the inflow and outflow ends of the prosthetic heart valve PHV flare radially outwardly relative to the axial center of the prosthetic heart valve PHV, with the flared portions generally wrapping or otherwise contouring to the inflow and outflow sides of the native valve annulus VA (which in this case is a native aortic valve annulus). The band 510 is preferably designed to perforate, tear, or otherwise break once the balloon 580 has reached a pre-determined pressure. For example, the band 510 may have a generally continuous ring shape, with a perforation provided to partially interrupt the continuity of the ring shape. In one example, a number of holes or apertures may be positioned in the band 510, each hole being positioned a spaced distance from an adjacent hole, with the holes being aligned with each other in a direction parallel to the longitudinal axis of the prosthetic heart valve PHV. These holes may create a preferential break line in the band 510, so that as the balloon 580 has been pressurized enough to begin forcing the flared ends of the prosthetic heart valve PHV to generally wrap or contour around the native valve annulus VA, the band 510 tears along the perforation line. It should be understood that, although one perforation line is described, multiple perforation lines may be provided. In other embodiments, perforations may be omitted and the band 510 may break simply when the expansion force of the balloon 580 exceeds the hoop strength of the band 510. In some embodiments, the band 510 is configured to break when the balloon 580 reaches a pressure of between about 3 atm and about 6 atm. In some embodiments, the band 510 may be formed of a thermoplastic resin or elastomer, such those offered under the trade names of Tecothane™ or ChronoPrene™.

As shown in FIGS. 4C and 4D, the axial center of the prosthetic heart valve PHV may be in contact with the native valve annulus VA when the band 510 breaks, or may be nearly in contact. As the band 510 breaks (shown in FIG. 4C), the band 510 stops limiting expansion of the balloon 580, and thus the axial center of the balloon 580 may rapidly expand if not already in contact with the native valve annulus VA. In other words, immediately before or after the band 510 breaks, the axial center of the prosthetic heart valve PHV is in contact with the native valve annulus VA, and the inflow and outflow ends of the prosthetic heart valve PHV wrap around or otherwise contour (e.g. flare radially outwardly) into contact with the inflow and outflow sides of the native valve annulus VA. The resulting positioning is shown in FIG. 4D, with the band 510 remaining trapped between the outside of the prosthetic heart valve PHV and the anatomy at the native valve annulus VA. In some embodiments, the band 510 may have one or more portions fixed to the prosthetic heart valve PHV, for example via a tack suture, to hold the band 510 in place after breaking so that the band 510 cannot enter the circulation after breaking.

Although the embodiments of band 510 described above refer to the band 510 breaking or otherwise perforating to allow for temporary constrictions, it should be understood that band 510 may provide a similar functionality without breaking, but rather expanding or stretching after a threshold pressure or force is applied. For example, the band 510 may be formed of urethane that is stretchable, without the need of the band 510 to break. In some embodiments, the band 510 may be formed of polyethylene or an elastomer offered under the trade name PRBAX®. In a stretchable band 510 embodiment, the band 510 may be configured to start stretching or expanding only after the prosthetic heart valve PHV flares from the balloon. For example, in FIG. 4B, the band 510 may not yet have any significant stretching or expansion, with the stretching or expansion beginning when additional force is applied by the balloon, as shown in FIGS. 4C-D.

As shown in FIG. 4D, the configuration of band 510 temporarily limiting expansion of the center portions of the prosthetic heart valve PHV and/or balloon 580 allows the prosthetic heart valve PHV to be contoured to wrap along the surfaces of the native valve annulus VA. This may help to reduce or eliminate the likelihood of PV leak occurring, for example because of the very close fit between the prosthetic heart valve PHV and native valve annulus VA. It should be understood that balloon-expandable prosthetic heart valves PHV are typically formed with stents having plastic expansion properties (e.g. stainless steel or cobalt chrome), so the prosthetic heart valve PHV will typically take the shape that the balloon and/or the patient anatomy force the prosthetic heart valve PHV to take. Thus, unlikely self-expanding prosthetic heart valves, balloon expandable prosthetic heart valves often have generally cylindrical shapes upon implantation, at least in part due to the balloons taking generally cylindrical shapes during expansion. The temporary limiting band 510 described above may instead allow for this flared shape of a balloon expandable prosthetic heart valve PHV, potentially reducing or eliminating PV leak following implantation.

Although FIGS. 4A-D are shown and described as a single band being used at an axial center of the balloon/valve assembly, it should be understood that more than one band may be used, and the one or more bands may be positioned differently than shown. In some embodiments, it may be desirable or sufficient to use a configuration of bands so that only the outflow end of the prosthetic heart valve PHV has the outward flare upon implantation, or that only the inflow end of the prosthetic heart valve PHV has the outward flare upon implantation.

An effect similar to that shown in FIGS. 4A-D may be provided with other mechanisms besides a band 510. For example, the frame or stent of the prosthetic heart valve PHV may be designed to have variable deployment characteristics. As one example, the strut geometry of the frame near the axial center may be different than the strut geometry near one or both terminal ends of the stent, with the frame near the axial center requiring more force to expand. Thus, as the balloon 580 expands, one or both terminal ends of the frame of the prosthetic heart valve PHV will begin to expand similar to that shown in FIG. 4B, with the different strut geometry near the axial center of the stent resisting expansion until additional force is applied from the balloon 580. In some examples, struts may be thicker near the axial center of the frame, and/or cells may have smaller areas near the axial center of the frame, in order to achieve greater resistance to expansion at the axial center portion of the frame compared to one or both terminal end portions of the frame. Further, although much of the present disclosure is directed to preventing or mitigating PV leak, the flared outflow shape of the prosthetic heart valve PHV, such as that shown in FIG. 4D, may alternately or additionally provide better hemodynamics than a comparable prosthetic heart valve that does not include such an outflow flare.

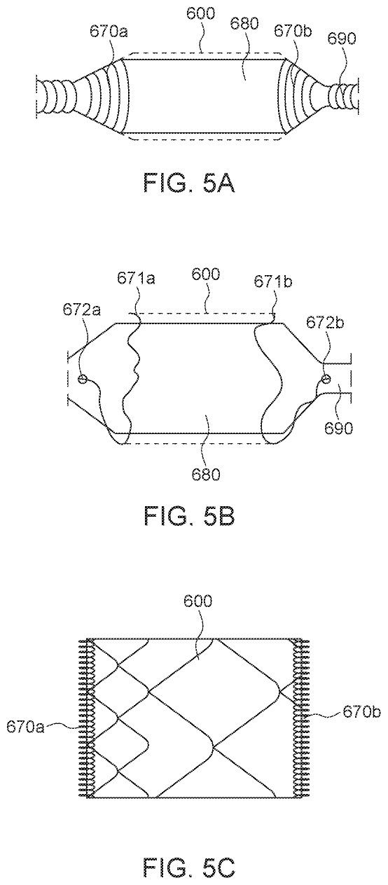

FIGS. 5A-C illustrate a prosthetic heart valve 600 at different stages of deployment via a balloon catheter 690, the prosthetic heart valve 600 including a PV leak mitigation feature in the form of an unravelling cuff. In particular, FIG. 5A illustrates prosthetic heart valve 600 in a collapsed or crimped condition over a deflated balloon 680 of a balloon catheter 690. The prosthetic heart valve 600 may take the form of any of the other prosthetic heart valves described herein, although it may include an additional component in the form of one or more unravelling cuffs. Thus, prosthetic heart valve 600 may include an inner cuff and/or outer cuff similar to those described in connection with FIGS. 1A-K, and the unravelling cuff(s) may be provided as additional PV leak mitigation feature, although it may be preferable to omit an outer cuff in the style of outer cuff 270 in relation to prosthetic heart valve 600.

Still referring to FIG. 5A, prosthetic heart valve 600 may include a first outer cuff 670a on an inflow end of the prosthetic heart valve 600, and a second outer cuff 670b on an outflow end of the prosthetic heart valve 600. In some embodiments, either first outer cuff 670a may be omitted, or second outer cuff 670b may be omitted. Unlike outer cuff 270, outer cuffs 670a-b are each in the form of a thread, cord, or other string that preferably has a plush or fuzzy texture. In some examples, the thread, cord, or other string may be in the form of a suture, and in some examples may twisted and fuzzy and may be formed of PET, PE, or UHMWPE. In some embodiments, the thread, cord, or other string may be formed as a multi-ply structure with a high filament count. Each outer cuff 670a, 670b may include a first end 671a, 671b that is coupled (e.g. tied or sutured) to the stent of the prosthetic heart valve 600. Each outer cuff 670a, 670b may extend to a second opposite end 672a, 672b that is reversibly fixed to balloon 680. The length of each outer cuff 670a, 670b extending between the two opposing ends may generally wrap around the circumference of the stent of the prosthetic heart valve 600, with portions weaving into and out of the cells (and/or wrapped around struts forming the cells) forming the stent of the prosthetic heart valve 600. In some embodiments, the outer cuffs 670a, 670b may each interweave with the frame between 1 and 10 times. While the prosthetic heart valve 600 is in the crimped condition, as shown in FIG. 5A, the excess length of each outer cuff 670a, 670b may wrap around the balloon 680, including at positions beyond the terminal ends of the prosthetic heart valve 600.

As shown in FIG. 5B, as fluid (e.g. a liquid, including saline) is passed through a lumen of the balloon catheter 690 and into balloon 680, the balloon 680 begins to expand, forcing the prosthetic heart valve 600 to expand. As this occurs, the outer cuffs 670a, 670b unravel, due to the circumference of the prosthetic heart valve 600 expanding. FIG. 5B illustrates the balloon 680 and prosthetic heart valve 600 in a partially expanded condition. As the balloon 680 reaches full inflation, and the prosthetic heart valve 600 reaches full expansion shown in FIG. 5C, the outer circumference of the prosthetic heart valve 600 becomes equal or substantially equal to the length of each outer cuff 670a, 670b, such that each outer cuff 670a, 670b forms a substantially circular thread at the respective inflow/outflow end of the prosthetic heart valve 600. The material of outer cuffs 670a, 670b, particularly if the material has a plush and/or fuzzy texture, will help mitigate or eliminate PV leak as the outer cuffs 670a, 670b are pressed into the native valve annulus upon full expansion of the prosthetic heart valve 600. Although some materials are described above as suitable examples for a plush and/or fuzzy material/texture, other suitable examples may include a multifilar polyester suture, polyurethane, nylon, or polyethylene. In some examples, depending on the profile of the balloon 680 when collapsed, the outer cuffs 670a, 670b may protrude outwardly far enough to act as an edge protector during delivery, particularly when the balloon 680 traverses the aortic arch. In other words, as the balloon 680 passes through the vasculature with a prosthetic heart valve mounted thereon, undesirable contact between a leading edge of the prosthetic heart valve and the anatomy (which might dislodge the prosthesis from the balloon 680) may be prevented due to the presence of one or both outer cuffs 670a, 670b. Stated in another way, if contact occurs between the leading edge of the balloon 680 and the patient's anatomy during anatomy, that contact may preferentially happen at one or both outer cuffs 670a, 670b due to their positioning, instead of such contact happening at the edge of the prosthetic heart valve.

Referring back to FIG. 5B, the second ends 672a, 672b of the outer cuffs 670a, 670b may be releasably coupled to the balloon 680 or to a nosecone of the delivery device. For example, the second ends 672a, 672b may be tacked or lightly trapped in the nosecone or at a proximal or distal end of the balloon bond region. With these configurations, when the prosthetic heart valve 600 is in the collapsed condition of FIG. 5A, only a very small portion of the length of the outer cuffs 670a, 670b needs to overlap the prosthetic heart valve 600, with at least half, at least two-thirds, or at least three-quarters of the length of the outer cuffs 670a, 670b being positioned beyond the respective end of the prosthetic heart valve 600. This allows for a relatively small bulk created from the outer cuffs 670a, 670b, especially compared to other outer cuffs that are always positioned directly over the stent of a prosthetic heart valve, whether collapsed or expanded. When the balloon 680 expands, the frame of the prosthetic heart valve will also expand, and the pressure may break the tack fixing the second ends 672a, 672b of the outer cuffs. In some embodiments, both ends of the thread or cord forming an outer cuff may be attached to the frame of the prosthetic heart valve, with the slack in the thread or cord pulled over the balloon and subsequently tightened during inflation, pulling the thread or cord into position for PV leak prevention or mitigation.

According to one aspect of the disclosure, a prosthetic heart valve system, comprises:
a balloon expandable prosthetic heart valve, the prosthetic heart valve having an inflow end portion, an outflow end portion, and a center portion between the inflow end portion and the outflow end portion; and
a delivery catheter having a balloon assembly on a distal end portion of the delivery catheter, the balloon assembly having a proximal portion, a distal portion, and a center portion between the proximal portion and the distal portion,
wherein, in a delivery condition of the system, the prosthetic heart valve is crimped over the balloon while the balloon assembly is deflated, and
wherein in a deployment condition of the system, the balloon assembly is inflated so that the distal portion of the balloon assembly has a diameter that is larger than a diameter of the center portion of the balloon assembly, and so that the outflow end portion of the prosthetic heart valve flares radially outwardly relative to the center portion of the prosthetic heart valve; and/or
the balloon assembly includes a first balloon and a second balloon; and/or
the second balloon is positioned within the first balloon, the second balloon being positioned only at the distal portion of the balloon assembly, and in the deployment condition of the system, the second balloon is inflated to a diameter that is larger than the diameter of the center portion of the balloon assembly; and/or
the first balloon is positioned within the second balloon, the second balloon being positioned only at the distal portion of the balloon assembly, and in the deployment condition of the system, the second balloon is inflated to a diameter that is larger than the diameter of the center portion of the balloon assembly; and/or
the balloon assembly includes only a single balloon; and/or
the single balloon has a uniform compliance along a length of the single balloon, the single balloon being contoured so that when the single balloon is inflated, a distal portion of the single balloon has a diameter that is larger than a diameter of a center portion of the single balloon; and/or
the single balloon has a proximal portion having a first compliance, and a distal portion having a second compliance less than the first compliance, so that when the single balloon is inflated while a center portion of the single balloon is positioned within a native valve annulus of a patient, a distal portion of the single balloon has a diameter that is larger than a diameter of the center portion of the single balloon; and/or
in the delivery condition of the system, a band circumscribes the center portion of the prosthetic heart valve; and/or
in the deployment condition of the system, the band limits expansion of the balloon assembly to maintain the diameter of the center portion of the balloon assembly; and/or
the system has a final implanted condition in which the band either (i) does not circumscribe the center portion of the prosthetic heart valve, the band being configured to break upon transition from the deployment condition to the final implanted condition; or (ii) does circumscribe the center portion of the prosthetic heart valve, the band being configured to stretch upon transition from the deployment condition to the final implanted condition.

According to a further aspect of the disclosure, a method of implanting a prosthetic heart valve comprises:
delivering a prosthetic heart valve to a native valve annulus while the prosthetic heart valve is crimped on a balloon assembly of a balloon catheter and the balloon assembly is deflated; and
inflating the balloon assembly to expand the prosthetic heart valve so that a center portion of the prosthetic heart valve contacts the native valve annulus and so that an inflow end portion of the prosthetic heart valve flares radially outwardly relative to the center portion of the prosthetic heart valve; and/or
inflating the balloon assembly includes inflating a first balloon of the balloon assembly to a first diameter, and inflating a second balloon of the balloon assembly to a second diameter greater than the first diameter, so that the second balloon forces the inflow end portion of the prosthetic heart valve to flare radially outwardly relative to the center portion of the prosthetic heart valve; and/or
the balloon assembly includes a single balloon having a uniform compliance, and inflating the balloon assembly includes inflating the single balloon to a pre-defined shape in which a distal end of the single balloon has a diameter that is larger than a center portion of the single balloon; and/or
the balloon assembly includes a single balloon having a proximal portion with a first compliance, and a distal portion with a second compliance less than the first compliance, and inflating the balloon assembly includes inflating the single balloon until the proximal portion presses the prosthetic heart valve into the native valve annulus, and continuing to inflate the balloon assembly so that the distal portion of the single balloon has a diameter that is larger than a diameter of the center portion of the single balloon; and/or
inflating the balloon assembly includes starting to inflate the balloon assembly while a band circumscribes the center portion of the prosthetic heart valve and a center portion of the balloon assembly, and continuing to inflate the balloon assembly so that a distal portion of the balloon assembly inflates to a diameter larger than a diameter of the center portion of the balloon assembly while the band still circumscribes the center portion of the prosthetic heart valve and the center portion of the balloon assembly; and/or
further inflating the balloon assembly until the band breaks and no longer circumscribes the center portion of the prosthetic heart valve and the center portion of the balloon assembly; and/or
the prosthetic heart valve includes a frame formed of struts having a strut geometry, the strut geometry of the frame at the center portion of the prosthetic heart valve being different than the strut geometry of the frame at the inflow end of the prosthetic heart valve such that more force is required to expand the frame at the center portion of the prosthetic heart valve than is required to expand the frame at the inflow portion of the prosthetic heart valve.

According to still another aspect of the disclosure, a prosthetic heart valve system comprises:
a prosthetic heart valve including a balloon expandable stent and a prosthetic valve assembly mounted within the stent;
a delivery catheter having a balloon on a distal end portion of the delivery catheter, the system having (i) a delivery condition in which the prosthetic heart valve is crimped over the balloon while the balloon is deflated, (ii) a partially deployed condition in which the prosthetic heart valve is partially expanded and the balloon is partially inflated, and (iii) a fully deployed condition in which the prosthetic heart valve is fully expanded and the balloon is fully inflated; and
a first outer cuff formed by a thread having a first end coupled to the balloon and a second end coupled to the stent, wherein in the delivery condition, the first outer cuff has a middle portion that wraps around the stent and wraps around a portion of the balloon positioned beyond a first end of the stent; and/or
as the system transitions from the delivery condition to the partially deployed condition to the fully deployed condition, the first outer cuff unravels; and/or
in the fully deployed condition of the system, a length of the first outer cuff is substantially equal to a circumference of the stent; and/or
a second outer cuff formed by a thread having a first end coupled to the balloon and a second end coupled to the stent, wherein in the delivery condition, the second outer cuff has a middle portion that wraps around the stent and wraps around a portion of the balloon positioned beyond a second end of the stent, the first outer cuff being positioned at an inflow end of the prosthetic heart valve and the second outer cuff being positioned at an outflow end of the prosthetic heart valve.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a balloon expandable prosthetic heart valve, the prosthetic heart valve having an inflow end portion, an outflow end portion, and a center portion between the inflow end portion and the outflow end portion; and
a delivery catheter having a balloon assembly on a distal end portion of the delivery catheter, the balloon assembly having a proximal portion, a distal portion, and a center portion between the proximal portion and the distal portion,
wherein, in a delivery condition of the system, the prosthetic heart valve is crimped over the balloon while the balloon assembly is deflated, and
wherein in a deployment condition of the system, the balloon assembly is inflated so that the distal portion of the balloon assembly has a diameter that is larger than a diameter of the center portion of the balloon assembly, and so that the outflow end portion of the prosthetic heart valve flares radially outwardly relative to the center portion of the prosthetic heart valve.

2. The prosthetic heart valve system of clam 1, wherein the balloon assembly includes a first balloon and a second balloon.

3. The prosthetic heart valve system of claim 2, wherein the second balloon is positioned within the first balloon, the second balloon being positioned only at the distal portion of the balloon assembly, and in the deployment condition of the system, the second balloon is inflated to a diameter that is larger than the diameter of the center portion of the balloon assembly.

4. The prosthetic heart valve system of claim 2, wherein the first balloon is positioned within the second balloon, the second balloon being positioned only at the distal portion of the balloon assembly, and in the deployment condition of the system, the second balloon is inflated to a diameter that is larger than the diameter of the center portion of the balloon assembly.

5. The prosthetic heart valve system of claim 1, wherein the balloon assembly includes only a single balloon.

6. The prosthetic heart valve system of claim 5, wherein the single balloon has a uniform compliance along a length of the single balloon, the single balloon being contoured so that when the single balloon is inflated, a distal portion of the single balloon has a diameter that is larger than a diameter of a center portion of the single balloon.

7. The prosthetic heart valve system of claim 5, wherein the single balloon has a proximal portion having a first compliance, and a distal portion having a second compliance less than the first compliance, so that when the single balloon is inflated while a center portion of the single balloon is positioned within a native valve annulus of a patient, a distal portion of the single balloon has a diameter that is larger than a diameter of the center portion of the single balloon.

8. The prosthetic heart valve system of claim 1, wherein in the delivery condition of the system, a band circumscribes the center portion of the prosthetic heart valve.

9. The prosthetic heart valve system of claim 8, wherein in the deployment condition of the system, the band limits expansion of the balloon assembly to maintain the diameter of the center portion of the balloon assembly.

10. The prosthetic heart valve system of claim 9, wherein the system has a final implanted condition in which the band either (i) does not circumscribe the center portion of the prosthetic heart valve, the band being configured to break upon transition from the deployment condition to the final implanted condition; or (ii) does circumscribe the center portion of the prosthetic heart valve, the band being configured to stretch upon transition from the deployment condition to the final implanted condition.

11. A method of implanting a prosthetic heart valve, the method comprising:
delivering a prosthetic heart valve to a native valve annulus while the prosthetic heart valve is crimped on a balloon assembly of a balloon catheter and the balloon assembly is deflated; and
inflating the balloon assembly to expand the prosthetic heart valve so that a center portion of the prosthetic heart valve contacts the native valve annulus and so that an inflow end portion of the prosthetic heart valve flares radially outwardly relative to the center portion of the prosthetic heart valve.

12. The method of claim 11, wherein inflating the balloon assembly includes inflating a first balloon of the balloon assembly to a first diameter, and inflating a second balloon of the balloon assembly to a second diameter greater than the first diameter, so that the second balloon forces the inflow end portion of the prosthetic heart valve to flare radially outwardly relative to the center portion of the prosthetic heart valve.

13. The method of claim 11, wherein the balloon assembly includes a single balloon having a uniform compliance, and inflating the balloon assembly includes inflating the single balloon to a pre-defined shape in which a distal end of the single balloon has a diameter that is larger than a center portion of the single balloon.

14. The method of claim 11, wherein the balloon assembly includes a single balloon having a proximal portion with a first compliance, and a distal portion with a second compliance less than the first compliance, and inflating the balloon assembly includes inflating the single balloon until the proximal portion presses the prosthetic heart valve into the native valve annulus, and continuing to inflate the balloon assembly so that the distal portion of the single balloon has a diameter that is larger than a diameter of the center portion of the single balloon.

15. The method of claim 11, wherein inflating the balloon assembly includes starting to inflate the balloon assembly while a band circumscribes the center portion of the prosthetic heart valve and a center portion of the balloon assembly, and continuing to inflate the balloon assembly so that a distal portion of the balloon assembly inflates to a diameter larger than a diameter of the center portion of the balloon assembly while the band still circumscribes the center portion of the prosthetic heart valve and the center portion of the balloon assembly.

16. The method of claim 15, further comprising further inflating the balloon assembly until the band breaks and no longer circumscribes the center portion of the prosthetic heart valve and the center portion of the balloon assembly.

17. The method of claim 11, wherein the prosthetic heart valve includes a frame formed of struts having a strut geometry, the strut geometry of the frame at the center portion of the prosthetic heart valve being different than the strut geometry of the frame at the inflow end of the prosthetic heart valve such that more force is required to expand the frame at the center portion of the prosthetic heart valve than is required to expand the frame at the inflow portion of the prosthetic heart valve.

18. A prosthetic heart valve system comprising:
a prosthetic heart valve including a balloon expandable stent and a prosthetic valve assembly mounted within the stent;
a delivery catheter having a balloon on a distal end portion of the delivery catheter, the system having (i) a delivery condition in which the prosthetic heart valve is crimped over the balloon while the balloon is deflated, (ii) a partially deployed condition in which the prosthetic heart valve is partially expanded and the balloon is partially inflated, and (iii) a fully deployed condition in which the prosthetic heart valve is fully expanded and the balloon is fully inflated; and
a first outer cuff formed by a thread having a first end coupled to the balloon and a second end coupled to the stent, wherein in the delivery condition, the first outer cuff has a middle portion that wraps around the stent and wraps around a portion of the balloon positioned beyond a first end of the stent.

19. The prosthetic heart valve system of claim 18, wherein as the system transitions from the delivery condition to the partially deployed condition to the fully deployed condition, the first outer cuff unravels.

20. The prosthetic heart valve system of claim 19, wherein in the fully deployed condition of the system, a length of the first outer cuff is substantially equal to a circumference of the stent.

21. The prosthetic heart valve system of claim 18, further comprising a second outer cuff formed by a thread having a first end coupled to the balloon and a second end coupled to the stent, wherein in the delivery condition, the second outer cuff has a middle portion that wraps around the stent and wraps around a portion of the balloon positioned beyond a second end of the stent, the first outer cuff being positioned at an inflow end of the prosthetic heart valve and the second outer cuff being positioned at an outflow end of the prosthetic heart valve.
